(19) United States
(12) Patent Application Publication
(10) Pub. No.: US 2023/0137976 A1
(43) Pub. Date: May 4, 2023

(54) INJECTION DEVICE WITH END OF DOSE INDICATOR

(71) Applicant: AbbVie Inc., North Chicago, IL (US)

(72) Inventors: Sean Corrigan, Chicago, IL (US); Bradley Gould, Evanston, IL (US); David T. Andersen, Des Plaines, IL (US); Kelly Burton, Chicago, IL (US); Tyler W. Mitchell, Chicago, IL (US); Ian Cleathero, Melton Mowbray (GB)

(21) Appl. No.: 17/896,983

(22) Filed: Aug. 26, 2022

Related U.S. Application Data
(63) Continuation of application No. 17/579,182, filed on Jan. 19, 2022, now abandoned.
Application No. 17/579,182 is a continuation of application No. 16/434,887, filed on Jun. 7, 2019, now abandoned.
(60) Provisional application No. 62/682,592, filed on Jun. 8, 2018.

(51) Int. Cl. A61M 5/315; A61M 5/20

(52) U.S. Cl. CPC A61M 5/3157; A61M 5/20; A61M 5/2053; A61M 5/2033

(57) ABSTRACT

An injection device includes a housing having an internal volume and a viewing portion. A movable seal is disposed in the internal volume and is movable from an initial position to an injection end position to urge a drug product out of the housing. An indicator assembly is disposed in the internal volume and includes a dose end indicator. The indicator assembly is biased from a dose remaining position toward a dose end position where the dose end indicator is visible through the viewing portion. A release mechanism releasably holds the indicator assembly in the dose remaining position and is linked to the movable seal such that the movable seal reaching the injection end position causes the release mechanism to release the indicator assembly to the dose end position.

### CROSS-REFERENCE TO RELATED APPLICATIONS

This application is a continuation of and claims priority to U.S. patent application Ser. No. 16/434,887, filed Jun. 7, 2019, which claims priority to U.S. Provisional Patent Application No. 62/682,592, filed Jun. 8, 2018, the disclosures of which are incorporated herein by reference in their entirety.

### TECHNICAL FIELD

The present disclosure relates to an injection device for injecting a drug product, such as a therapeutic agent, into a patient.

### BACKGROUND

One of the most common routes of administration for drug products is by injection, such as intravenous, subcutaneous, or intramuscular injection. A syringe containing a drug product is often used for the injection, which is often carried out by trained medical personnel. In certain instances, a patient is trained in the use of the syringe to allow for self-injection. Moreover, certain drug products are formulated in pre-filled syringes for patient use, to avoid the need for the patient to fill the syringe. Some patients, however, may be averse to carrying out self-injection with a manual syringe, particularly if the patient lacks the dexterity to manipulate the syringe to fill the syringe and inject the drug product from the syringe. Automatic injection devices offer an alternative to a manual syringes for delivering a drug product, as automatic injection devices are easier to use.

### SUMMARY

The present disclosure provides injection devices with an end of dose indicator assembly that is simultaneously viewable from at least two different directions. The end of dose indicator assembly may be a binary indicator.

In an embodiment, the present disclosure provides an injection device including a housing having an internal volume and a viewing portion. A movable seal is disposed in the internal volume and is movable from an initial position to an injection end position to urge a drug product out of the housing. An indicator assembly is disposed in the internal volume and includes a dose end indicator. The indicator assembly is biased from a dose remaining position toward a dose end position where the dose end indicator is visible through the viewing portion. A release mechanism releasably holds the indicator assembly in the dose remaining position and is linked to the movable seal such that the movable seal reaching the injection end position causes the release mechanism to release the indicator assembly to the dose end position.

### BRIEF DESCRIPTION OF THE FIGURES

The foregoing and other objects, features and advantages of the exemplary embodiments will be more fully understood from the following description when read together with the accompanying drawings, in which:

FIG. 1A illustrates an exemplary injection device including an indicator assembly in a dose remaining position according to an example embodiment.

FIG. 1B is a perspective view of the injection device illustrated in FIG. 1A when a dose end indicator of the indicator assembly is visible through a viewing portion of a housing of the injection device.

FIG. 2A is a cross-sectional view of the injection device illustrated in FIG. 1A taken along line 2A-2A while a release mechanism releasably holds an indicator assembly in a dose remaining position.

FIG. 2B is a cross-sectional view of the injection device illustrated in FIG. 1B taken along line 2B-2B as a movable seal moves to an injection end position such that the release mechanism releases the indicator assembly to a dose end position.

FIG. 3A illustrates another exemplary injection device that incorporates a firing button according to an example embodiment.

FIG. 3B is a cross-sectional view of the injection device illustrated in FIG. 3A taken along line 3B-3B.

FIG. 3C is a perspective view of the injection device illustrated in FIGS. 3A and 3B when the indicator assembly has reached a dose end position.

FIG. 4A illustrates another exemplary injection device that incorporates a firing button and an indicator assembly with a visual indicator that is a different color than a dose end indicator.

FIG. 4B is a perspective view of the injection device of FIG. 4A after the indicator assembly has been released to a dose end position such that the dose end indicator is visible.

FIG. 5A illustrates another exemplary injection device including an indicator assembly in a dose remaining position according to an example embodiment.

FIG. 5B is a cross-sectional view of the injection device illustrated in FIG. 5A taken along line 5B-5B.

FIG. 5C is a perspective view of the injection device illustrated in FIGS. 5A and 5B after the indicator assembly has been released to a dose end position such that a dose end indicator is visible.

FIG. 6A illustrates another exemplary injection device that has an indicator assembly with a visual indicator that is a different color than a dose end indicator.

FIG. 6B is a perspective view of the injection device of FIG. 6A after the indicator assembly has been released to a dose end position such that the dose end indicator is visible.

FIG. 7 illustrates an exemplary anchor for linking a movable seal to a tensioner according to an example embodiment.

FIG. 8A illustrates yet another exemplary injection device that includes an indicator assembly and utilizes pressurized fluid to drive an injection according to an example embodiment.

FIG. 8B is a perspective view of an exemplary septum that may be incorporated in the injection device illustrated in FIG. 8A to fluidly isolate two fluid spaces within the injection device according to an example embodiment.

FIG. 8C is a perspective view of another exemplary septum that may be incorporated in the injection device illustrated in FIG. 8A to fluidly isolate two fluid spaces within the injection device according to an example embodiment.

FIG. 8D is a cross-sectional view of an exemplary seal that may be incorporated in the injection device illustrated in FIG. 8A to fluidly isolate two fluid spaces within the injection device according to an example embodiment.

FIG. 9A is a cross-sectional view of an exemplary pin that allows progressive movement of an indicator assembly to a dose end position.

FIG. 9B is a cross-sectional view of an exemplary stepped pin that allows step-wise movement of an indicator assembly to a dose end position.

### DETAILED DESCRIPTION

The present disclosure provides injection devices with an indicator assembly including a dose end indicator viewable from at least both a side and a top of the injection device for indicating when an injection is completed. The indicator assembly is disposed in a housing of the injection device and is biased from a dose remaining position toward a dose end position where the dose end indicator is simultaneously visible from two or more different viewing positions through a viewing portion of the housing. A release mechanism releasably holds the indicator assembly in the dose remaining position. A movable seal that is linked to the release mechanism moves to an injection end position to release the indicator assembly into the dose end position.

The devices presented herein can be used for injecting a variety of drug products into a patient. In one embodiment, the injection device can be configured in the form of an automatic injection device. In some embodiments, the automatic injection device is configured in the form of a pen, i.e., a portable autoinjector that enables an individual to administer a dosage of a drug product. In other embodiments, the injection device can be configured as a traditional syringe or other type of injection device for injecting drug products.

As used herein, an “automatic injection device” (or “autoinjector”) is intended to refer to a device that enables an individual (also referred to herein as a user or a patient) to self-administer a dosage of a drug product. The automatic injection device differs from a standard syringe by the inclusion of a mechanism for automatically inserting the needle at an injection site, automatically delivering the drug product to the individual by injection, and automatically retracting the needle from the injection site when the mechanism is engaged.

As used herein, the term “drug product” refers to a composition intended for use in medical diagnosis, cure, treatment, or prevention of disease. A drug product may be a therapeutic agent or a combination of therapeutic agents. A drug product may include a therapeutic protein, for example, a peptide or antibody, or antigen-binding portion thereof. A drug product may include an anesthetic, steroid, and/or any other therapeutic agent(s). In one embodiment, a drug product represents a mixture of two or even more pharmacologically active agents. In some embodiments, the drug product is a liquid therapeutic agent which includes one or more biological agents, such as a protein, or antibody. For example, the liquid therapeutic agent can be a monoclonal antibody targeting interleukin 23A, such as risankizumab. In yet another example embodiment, the liquid therapeutic agent may comprise an antibody drug conjugate (ADC). Additional details regarding possible therapeutic agents, including are provided in U.S. Pat. No. 8,679,061, the contents of which are incorporated by reference herein in its entirety. The drug product may have any suitable volume, for example between about 0.5 ml and about 4 ml, e.g. about 2.25 ml.

As used herein, the term “proximal” refers to the portion or end of an injection device or component in the injection device furthest from an injection site of the user when the device is held against the person for an injection.

As used herein, the term “distal” refers to the portion or end of an injection device or a component of the injection device closest to an injection site of the user during an injection.

As used herein, the term “dose end” or “end of dose” refers to a movable seal, for example, a bung or a piston reaching a termination point within the barrel of a syringe following compression of a drug product through an aperture of the syringe to deliver a volume of the drug product for treatment of a patient for which the drug product is indicated.

The present disclosure provides injection devices with a binary end-of-dose (“EOD”) indicator that does not deploy until the injection is complete. In some embodiments, the indicator is incorporated in a single-use automatic injection device to alert a user that the injection is complete, and that the injection device may be discarded. In some embodiments, the indicator is incorporated in a re-usable injection device, such as an infusion pump, to indicate that a complete dose has been delivered and the user may, for example, replace a drug product container holding the dose.

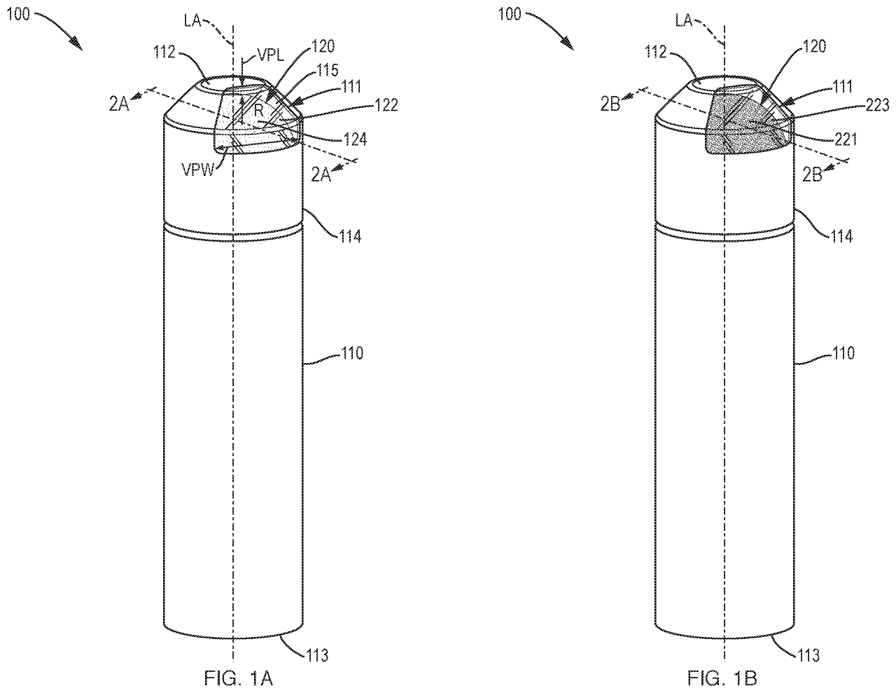

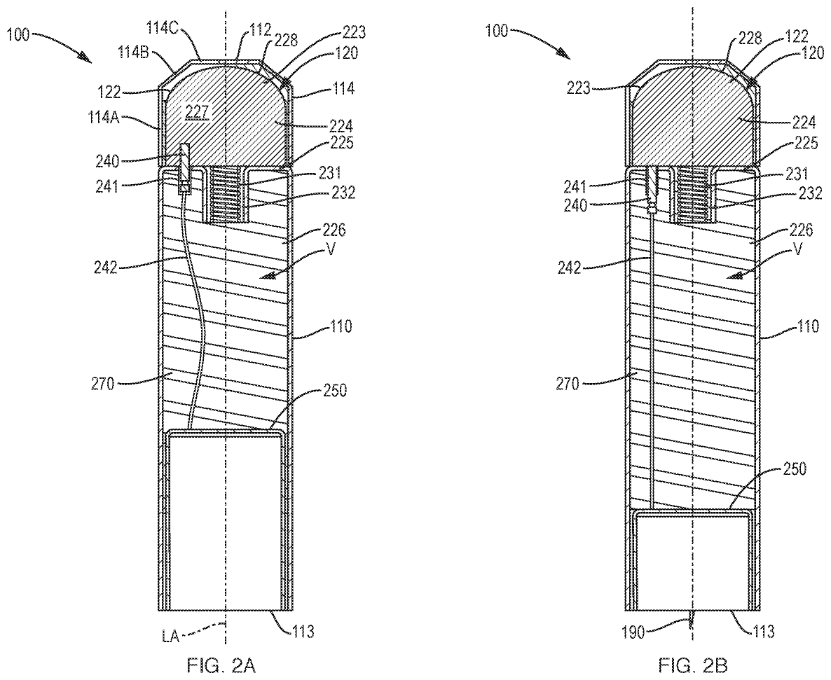

Referring now to the drawings, and more particularly to FIGS. 1A-2B, an exemplary embodiment of an injection device 100 is illustrated that includes a housing 110 having a viewing portion 111 and an internal volume V. In some embodiments, the housing 110 has a generally cylindrical shape with a proximal end 112 and a distal end 113 opposite the proximal end 112. The viewing portion 111 may comprise a transparent portion of the housing 110 to allow a user to view an indicator assembly 120 that is disposed inside the housing 110 and is aligned with the viewing portion 111. In some embodiments, the viewing portion 111 may be formed adjacent to the proximal end 112 in a cap 114 of the housing 110 through at least three viewing surfaces 114A, 114B, 114C of the cap 114 to allow a user to view the indicator assembly 120 while holding the injection device 110 in hand. As illustrated, each of the viewing surfaces 114A, 114B, 114C may define a separate viewing plane such that the viewing portion 111, when formed through the viewing surfaces 114A, 114B, 114C, may be visible from a variety of different angles and orientations.

In some embodiments, the injection device 100 may not include a cap. In such embodiments the viewing portion 111 may be formed in the proximal end 112 of the housing 110 through at least three viewing surfaces 114A, 114B, 114C to allow a user to view the indicator assembly 120 while holding the injection device 110 in hand. The same principles of the viewing portion 111 described with respect to the cap 114 are equally applicable to an embodiment of the injection device 100 formed without a cap.

The viewing portion 111 may be formed as an aperture or other opening in the housing 110 that is partially or fully covered by a viewing window 115. In some embodiments, the viewing window 115 is a generally translucent material and substantially transparent to allow a user to clearly view the indicator assembly 120 through the viewing portion 111. The viewing portion 111 may be formed, for example, about a longitudinal axis LA of the housing 110 extending through the proximal end 112 and the distal end 113. In some embodiments, the viewing portion 111 has a viewing portion width VPW that extends on an arc that is defined between approximately 1° and approximately 180° about the longitudinal axis LA to allow visibility through the viewing portion 111 from a variety of angles. In some embodiments, the viewing portion 111 has a viewing portion length VPL that is equal to a radius R of the housing 110, relative to the longitudinal axis LA.

A movable seal 250, for example a piston or a bung in FIGS. 2A and 2B, is disposed in a container, such as a barrel of a syringe or a cartridge disposed in the internal volume V of the housing 110. While the movable seal 250 is illustrated as a rodless piston, in some embodiments the movable seal 250 is connected to a plunger rod or similar element that translates the movable seal 250 along the longitudinal axis LA of the housing 110. In some embodiments, the movable seal 250 is a sealed end of a container that urges drug product out of the container during movement. It should thus be appreciated that the movable seal 250 may take many forms to urge drug product out of the housing 110 in accordance with the present disclosure.

The movable seal 250 is movable from an initial position, illustrated in FIG. 2A, to an injection end position, which is illustrated in FIG. 2B and described further herein, to urge drug product out of the housing 110. In some embodiments, the injection device 100 may include, for example, a biasing element 270, such as a coil spring, elastomeric material, and/or pressurized liquid or gas containers. The biasing element 270 may be directly coupled or linked, or indirectly coupled or linked via a plunger rod or other element, to the movable seal 250 such that, when activated, the biasing element 270 forces the movable seal 250 from the initial position to the injection end position to urge drug product out of the housing 110. In some embodiments, translation of the movable seal 250 along the longitudinal axis LA pressurizes drug product in the syringe barrel, or other container, to urge the drug product out of the syringe barrel.

With further reference to FIGS. 1A-2B, it is illustrated that the indicator assembly 120 is disposed inside the internal volume V, adjacent to the cap 114 and the proximal end 112 of the housing 110, and biased from a dose remaining position, illustrated in FIGS. 1A and 2A, toward a dose end position, illustrated in FIGS. 1B and 2B, where a dose end indicator 221 is visible through the viewing portion 111 of the housing 110. In some embodiments, the dose end indicator 221 is not a component of a plunger rod and/or provides end of dose indication independent of movement of any plunger rod.

In some embodiments, the indicator assembly 120 includes a dome 224 with a first portion 122 and a second portion 223, with each portion 122, 223 being a respective half of the dome 224. The first portion 122 is visible through the viewing portion 111 when the indicator assembly 120 is in the dose remaining position and the second portion 223, which includes the dose end indicator 221, is visible through the viewing portion 111 when the indicator assembly 120 is in the dose end position. In some embodiments, some of the first portion 122 may also be visible through the viewing portion 111 when the indicator assembly 120 is in the dose end position. The first portion 122 may include a visual indicator 124 that is visually distinct from the dose end indicator 221. The visual indicator 124 may be a first color or other type of visual marking, such as a symbol, that is visually distinct from the dose end indicator 221. The visual indicator 124 may be, for example, a light or white colored portion while the dose end indicator 221 is a dark color, such as black, red, etc., that is visually distinct from the visual indicator 124. While the visual indicator 124 is illustrated as having a first color and the dose end indicator 221 is illustrated as having a second color that is different from the first color, in some embodiments the visual indicator 124 may be a symbol or text, such as an X or the phrase “dose remaining,” while the dose end indicator 221 is a different symbol or text that conveys that the dose has ended, such as a checkmark or the phrase “dose completed.” It should thus be appreciated that there are many different ways in which the visual indicator 124 may convey that there is at least some dose of drug product remaining and the dose end indicator 221 may convey to a user that the dose has been delivered.

In some embodiments, the first portion 122 and the second portion 223 of the dome 224 may be integral or otherwise connected with one another to move together, i.e., static relative to one another, so movement of the dome 224 causes movement of both of the portions 122, 223. In some embodiments, the indicator assembly 120 may be disposed on one side of a partition 225 disposed in the internal volume V to separate the indicator assembly 120 from a cavity 226 in the housing 110 that houses the movable seal 250 and other components of the injection device 100.

In some embodiments, an indicator assembly biaser 231, for example, illustrated as a torsion spring, bears on the indicator assembly 120 to bias the indicator assembly 120 from the dose remaining position toward the dose end position. In some embodiments, the biaser 231 is placed in an indicator assembly biaser cavity 232 formed adjacent to the partition 225. The biaser 231 may store, for example, rotational energy in a constrained position that, when released, pivots a portion or all of the indicator assembly 120 about the longitudinal axis LA, which thus defines a pivoting axis of the indicator assembly 120. While the exemplary embodiment illustrated in FIGS. 1A-2B includes the biaser 231 to pivotably bias the indicator assembly 120 from the dose remaining position toward the dose end position, it should be appreciated that the indicator assembly 120 may be naturally biased toward the dose end position without the need of a separate element. For example, the dome 224 may comprise an elastic material, such as a rubber element, that is twisted to the dose remaining position and naturally biased to untwist from the dose remaining position to the dose end position unless held in the twisted position.

Referring specifically now to FIGS. 2A and 2B, a release mechanism 240 is illustrated that releasably holds the indicator assembly 120 in the dose remaining position and is linked to the movable seal 250. In some embodiments, the release mechanism 240 is in the form of a pin, catch, or other element that extends through a release mechanism opening 241 formed in the partition 225 so the release mechanism 240 extends into the cavity 226 while also contacting some or all of the indicator assembly 120 to hold the indicator assembly 120 in the dose remaining position. To hold the indicator assembly 120 in the dose remaining position, the release mechanism 240 may abut against a contact surface 227 of the dome 224 and prevent free pivoting of the dome 224. In some embodiments, the release mechanism 240 is pressed into sides of the release mechanism opening 241 by the natural bias of the dome 224 and the contact surface 227 toward the dose end position. While the release mechanism 240 is illustrated and described as a pin that abuts against a surface 227 of the indicator assembly 120 to hold the indicator assembly 120 in the dose remaining position, in some embodiments the release mechanism 240 is formed as, for example, a hook or other type of similar element having a portion placed in an opening of the indicator assembly 120, or otherwise coupled to the indicator assembly 120, to releasably hold the indicator assembly 120 in the dose remaining position.

The release mechanism 240 may be coupled or linked directly or indirectly to the movable seal 250 by, for example, a tensioner 242, illustrated as a monofilament fiber or wire. In some embodiments, the tensioner 242 is slacked when the movable seal 250 is in the initial position, as illustrated in FIG. 2A, such that little, if any, tension applies to the release mechanism 240 through the tensioner 242. When the movable seal 250 translates along the longitudinal axis LA, as illustrated in FIG. 2B, the tensioner 242 becomes taut such that movement of the movable seal 250 pulls the release mechanism 240 along the longitudinal axis LA. In some embodiments, the tensioner 242 extends through a guide opening, or has another element associated therewith, and prevents the tensioner 242 from becoming tangled during operation.

In some embodiments, a needle shroud or other element may also be directly or indirectly coupled to or linked to the movable seal 250, independently of or dependently on linkage of the indicator assembly 120 to the movable seal 250, and deploys when the movable seal 250 reaches the injection end position. In this manner, the needle shroud may be deployed in temporal sequence with the dose end position of indicator assembly. Many types of needle shrouds are conventional and may be readily incorporated in the injection device 100. Similarly, the injection device 100 may include a syringe carrier holding a syringe that translates along the longitudinal axis LA with the movable seal 250 to urge drug product out of the housing 110. Many types of syringe carriers are conventional and may be readily incorporated in the injection device 100.

A stop 228, illustrated as a surface, is positioned in the internal volume V adjacent to the indicator assembly 120 such that the stop 228 prevents the indicator assembly 120 from moving past the dose end position. For example, the stop 228 may be placed in a rotation path of the dome 224 such that the second portion 223 hits the stop 228 when the indicator assembly 120 reaches the dose end position, with the abutment of the second portion 223 against the stop 228 preventing the dome 224 from further rotating.

To use the injection device 100, a user depresses or otherwise actuates an element, such as a firing button or plunger or a component at a distal end 113 of the injector (e.g. a distal end of a needle shroud), so the movable seal 250 translates along the longitudinal axis LA toward the distal end 113 to urge drug product out of the housing 110 through, for example, a needle 190. As the movable seal 250 translates, or otherwise moves, the tensioner 242 linking the release mechanism 240 to the movable seal 250 becomes taut, if not already taut, and allows the movable seal 250 to pull the release mechanism 240 toward the distal end 113. When the movable seal 250 reaches the injection end position in which the movable seal 250 has fully moved to urge drug product out of the housing 110, the release mechanism 240 releases the indicator assembly 120 to the dose end position by, for example, pulling out of the release mechanism opening 241 and thus out of contact with the contact surface 227. When the release mechanism 240 releases the indicator assembly 120, the bias on the indicator assembly 120 is configured to move the indicator assembly 120 to the dose end position where the dose end indicator 221 is visible through the viewing portion 111 of the housing 110, alerting a user that the dose has ended. The indicator assembly 120 contacting the stop 228, or another element, upon reaching the dose end position may be configured to provide a vibration, or other type of tactile feedback, in the housing 110. The indicator assembly 120 contacting the stop 228, or another element, upon reaching the dose end position may also, or alternatively, may be configured to provide a “click” or other type of audible feedback. While the movement of the movable seal 250 is illustrated as pulling the release mechanism 240, in some embodiments the movable seal 250 reaching the injection end position pushes, or causes pushing of, the release mechanism 240 to release the indicator assembly 120.

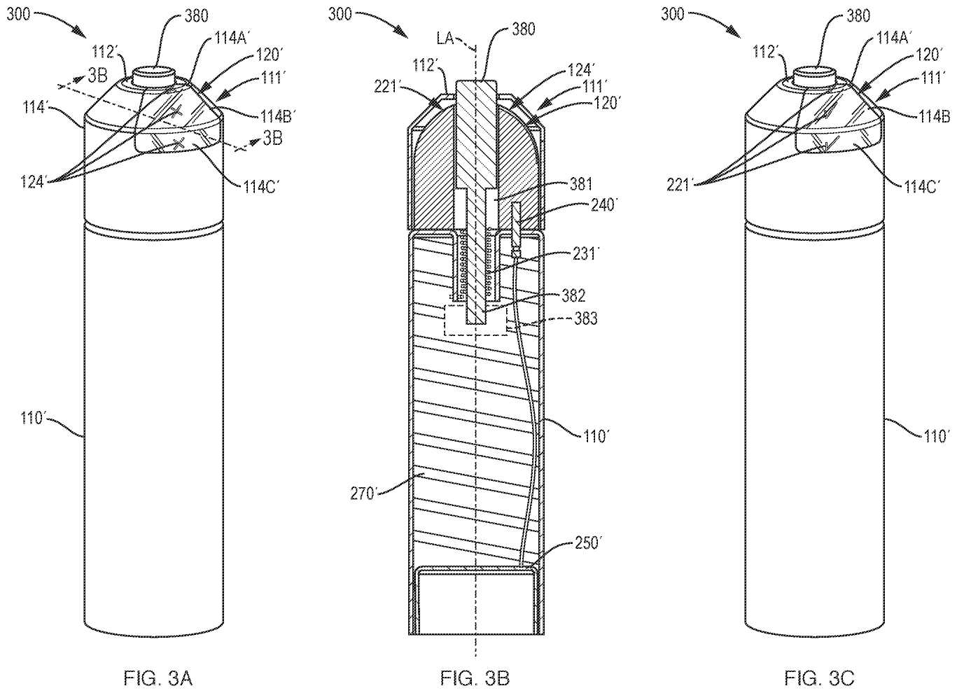

Referring now to FIGS. 3A, 3B, and 3C, another embodiment of the injection device 100, hereafter referred to as automatic injection device 300, is illustrated that incorporates a firing button 380 to activate an automatic injection by the automatic injection device 300. For elements of the automatic injection device 300 that are similar to elements of the automatic injection device 100, similar reference numerals are used with a prime (′) designation, e.g., housing 110′. As illustrated in FIG. 3B, the automatic injection device 300 has a modified injection assembly 120′ with a firing button opening 381 formed therein to accommodate the firing button 380, which extends through the firing button opening 381. The firing button 380 includes a button rod 382 surrounded by a biaser 231′ and is connected to a trigger assembly 383 that holds a biasing element 270′ in a constrained position until activated by depressing the firing button 380. The trigger assembly 383 releases the biasing element 270′ when the firing button 380 depresses, allowing the biasing element 270′ to deploy and start the automatic injection. In some embodiments, the trigger assembly 383 includes a lock or other element to prevent inadvertent triggering of an injection.

During the injection, a movable seal 250′ translates along the longitudinal axis LA to urge drug product out of the housing 110′. When the movable seal 250′ translates along the longitudinal axis LA to the injection end position, the release mechanism 240′ releases the injection assembly 120′. After the release mechanism 240′ releases the injection assembly 120′, the injection assembly 120′ rotates about the firing button 380 from the dose remaining position to the dose end position where one or more dose end indicators 221′, rather than one or more visual indicators 124′, are visible through the viewing portion 111′. As illustrated in FIGS. 3A and 3C, the visual indicators 124′ and the dose end indicators 221′ may be visually distinct symbols, illustrated as X's and check marks, respectively. In some embodiments, the number of visual indicators 124′ and dose end indicators 221′ may be at least equal to the number of viewing surfaces 114A′, 114B′, 114C′ of a cap 114′ of the housing 110′ so at least one visual indicator 124′ and/or at least one dose end indicator 221′ is visible through each of the viewing surfaces 114A′, 114B′, 114C′. Thus, the firing button 380 provides a relatively easy-to-use mechanism for automatically injecting drug product with the automatic injection device 300 and, once the injection is complete, causing the release mechanism 240′ to release the injection assembly 120′. In all other respects, the automatic injection device 300 may be similar to the injection device 100.

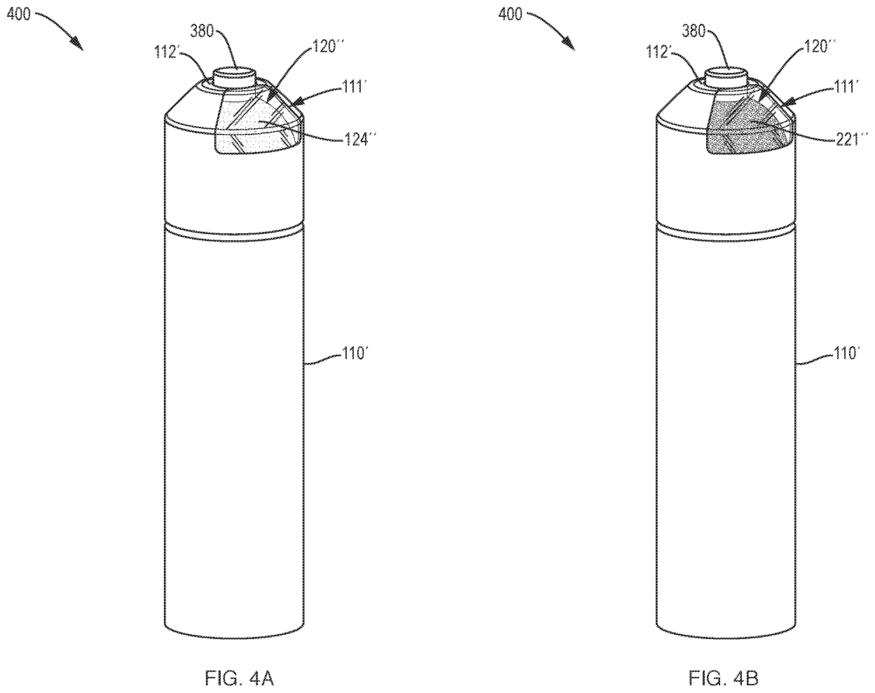

In another exemplary embodiment illustrated in FIGS. 4A and 4B, an automatic injection device 400 is provided that is similar to the automatic injection device 300, with similar elements numbered similarly in FIGS. 4A and 4B, but includes an indicator assembly 120″ with a visual indicator 124″ that is a different color from a dose end indicator 221″ of the indicator assembly 120″. In all other respects, the automatic injection device 400 is similar to the automatic injection device 300.

The injection devices 100, 300, 400 disclosed herein provide an indicator assembly 120, 120′, 120″ with a dose end indicator 221, 221′, 221″ that becomes visible through the viewing portion 111, 111′, 111″ when the movable seal 250, 250′ reaches the injection end position. In this sense, the indicator assembly 120, 120′, 120″ is a binary indicator that conveys that the movable seal 250, 250′ has reached the injection end position, which generally corresponds to the injection ending and the dose of drug product being urged out of the housing 110, 110′, via the release mechanism 240, 240′ releasing the indicator assembly 120, 120′, 120″ to the dose end position when the movable seal 250, 250′ reaches the injection end position. In some embodiments, such an indicator assembly 120, 120′, 120″ may forego so-called “dose progress indicators,” which indicate how much dose is remaining in the injection and may be confusing to users. The viewing portion 111, 111′ and the indicator assembly 120, 120′, 120″ may also be placed adjacent to the proximal end 112, 112′ of the injection device 100, 300, 400, rather than a middle portion of the device 100, 300, 400, so the indicator assembly 120, 120′, 120″ is simultaneously viewable from a variety of viewing directions and angles while performing the injection. In some embodiments, the indicator assembly 120, 120′, 120″ is viewable from at least two different viewing directions. Further, the release mechanism 240, 240′ releasably holding the indicator assembly 120, 120′, 120″ may be linked to the movable seal 250, 250′ such that the release mechanism 240, 240′ releases the indicator assembly 120, 120′, 120″ with little, if any, detrimental effect, such as friction sticking of the movable seal 250, 250′, on the movement of the movable seal 250, 250′ to the injection end position.

In some embodiments, the injection device 100, 300, 400 may include one or more other indicators in addition to the indicator assembly 120, 120′, 120″. For example, the injection device 100, 300, 400 may include an additional indicator that “clicks” or otherwise indicates when the injection has finished and the dose of drug product has been urged out of the housing 110, 110′. The additional indicator may be, for example, a button or other type of element that pops out of the housing 110, 110′ when the injection finishes. Thus, the additional indicator may provide a tactile and/or audible indication to alert a user that the injection has finished.

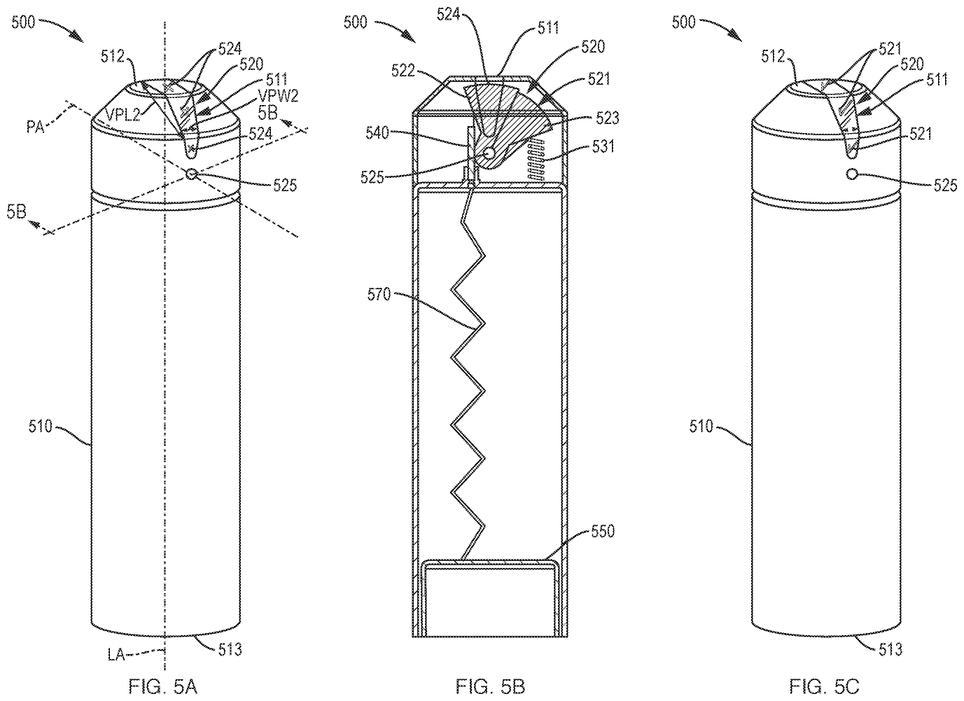

With reference now to FIGS. 5A, 5B, and 5C, another exemplary embodiment of an injection device 500 is illustrated that is similar to the injection device 100 but has an indicator assembly 520 that pivots to the dose end position about a pivoting pin 525 defining a pivot axis PA that is orthogonal relative to a longitudinal axis LA of the injection device 500. While the pivot axis PA is illustrated as being orthogonal relative to the longitudinal axis LA, in some embodiments the pivot axis PA is transverse, but not necessarily orthogonal, to the longitudinal axis LA. As illustrated, the pivoting pin 525 may extend through an outer surface of a housing 510 of the injection device 500, which may be generally cylindrical with a proximal end 512 and a distal end 513 opposite the proximal end 512. The housing 510 includes a viewing portion 511 that may be formed with a viewing portion width VPW2 that is relatively narrow compared to a viewing portion length VPL2 of the viewing portion 511. In other respects, the housing 510 may be similar to the previously described housing 110.

In some embodiments, the indicator assembly 520 is biased by a biaser 531, illustrated as a compression spring, from a dose remaining position, in which one or more visual indicators 524, illustrated as X's, of a first portion 522 of the indicator assembly 520 are visible through the viewing portion 511 as illustrated in FIG. 5A, to a dose end position in which one or more dose end indicators 521, illustrated as check marks, of a second portion 523 are visible through the viewing portion 511 as illustrated in FIG. 5C. As illustrated in FIGS. 5A and 5C, the visual indicator 524 may be a visually distinct symbol from the dose end indicator 521, as previously described. A release mechanism 540, illustrated as a pin, releasably holds the indicator assembly 520 in the dose remaining position and is linked to a movable seal 550 such that the movable seal reaching an injection end position causes the release mechanism 540 to release the indicator assembly 520 to the dose end position. The release mechanism 540 may be linked to the movable seal 550, for example, by a tensioner 570, illustrated as a flat or round cable, that unfolds and becomes taut as the movable seal 550 advances toward the injection end position, pulling the release mechanism 540 out of contact with, and thus releasing, the indicator assembly 520. When the release mechanism 540 releases the indicator assembly 520, the biaser 531 is unconstrained and forces the first portion 522 and the second portion 523, which may be static relative to one another, to pivot about the pivot axis PA so the dose end indicator 521 is visible through the viewing window 511. In other respects, the injection device 500 may be similar to the previously described injection device 100.

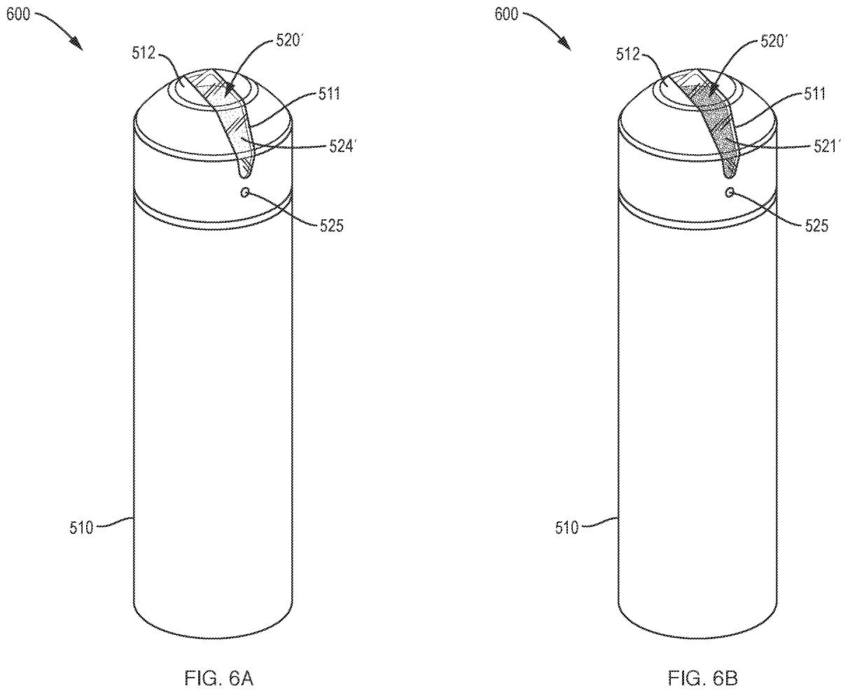

In another exemplary embodiment illustrated in FIGS. 6A and 6B, an automatic injection device 600 is provided that is similar to the automatic injection device 500, with similar elements numbered similarly in FIGS. 6A and 6B, but includes an indicator assembly 520′ with a visual indicator 524′ that is a different color from a dose end indicator 521′ of the indicator assembly 520′. The visual indicator 524′ of FIG. 6A may be, for example, a light or white colored portion while the dose end indicator 521′ of FIG. 6B is a dark color, such as black, red, etc., that is visually distinct from the visual indicator 524′. The visual indicator 524′ may convey that there is at least some dose of drug product remaining and the dose end indicator 521′ may convey to a user that the dose has been delivered. In all other respects, the automatic injection device 600 is similar to the automatic injection device 500.

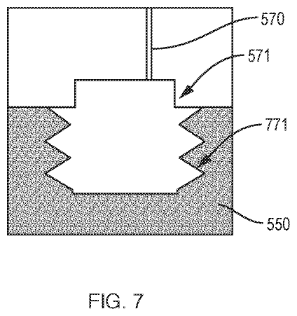

In some embodiments, and referring now to FIGS. 5B and 7, an anchor 571 may be configured to directly or indirectly couple to the movable seal 550 and may be connected to the tensioner 570 to link the movable seal 550 to the release mechanism 540. The anchor 571 may couple to the movable seal 550 by, for example, threading into threads 771 of the movable seal 550. The anchor 571 may be bonded to the tensioner 570 by an adhesive or otherwise connected to the tensioner 570 so the anchor 571 can pull the tensioner 570 and connected release mechanism 540 as the movable seal 550 moves toward the injection end position to urge drug product out of the housing 410.

In some embodiments a first portion of an indicator assembly is movable relative to a second portion of the indicator assembly. For example, one of the portions may be viewable through the viewing portion of the housing when the indicator assembly is in the dose remaining position. When the indicator assembly reaches the dose end position, one of the portions may move out of, or alternatively into, view through the viewing portion to indicate that the dose has been ejected from the housing.

From the foregoing, it should be appreciated that the injection devices 500, 600 provide a binary end of dose indicator assembly 520, 520′ that can convey to users that a dose of drug product has been injected without indicating progress of the injection. By having the indicator assembly 520, 520′ pivoted about the pivot axis PA extending orthogonally, or in some embodiments transversely, to the longitudinal axis LA, the indicator assembly 520, 520′ can be formed with a relatively small size. The relatively small size of the indicator assembly 520, 520′ may assist with, for example, assembly of the injection device 500, 600 and allow other components to fit within the injection devices 500, 600.

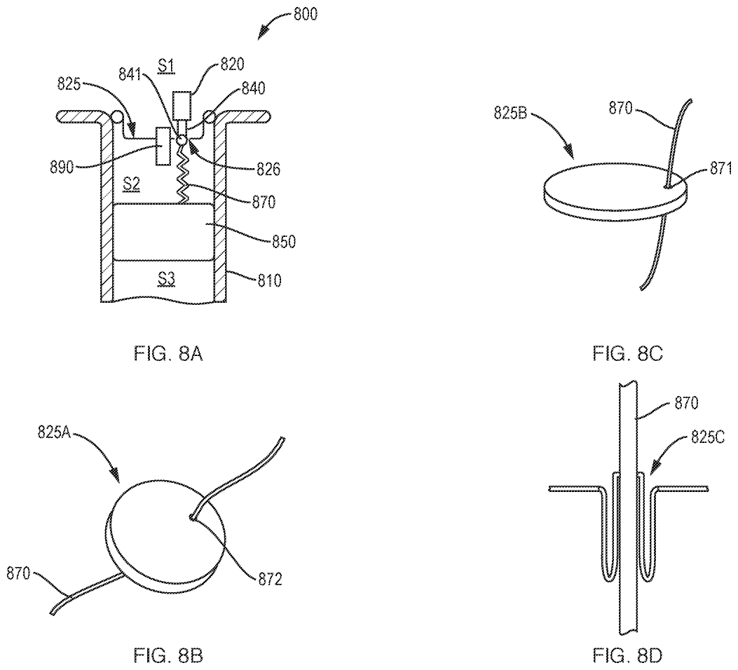

Referring now to FIG. 8A, a portion of an injection device 800 is illustrated that utilizes fluid pressure to drive a movable seal 850, such as a stopper, in a housing 810, rather than a spring or other type of solid biasing element. As used herein, a “fluid” may be, but is not limited to, a liquid, a gas, or a combination of a liquid and a gas. The movable seal 850 is linked to a release mechanism 840, such as a pin, that extends through a partition 825 that fluidly isolates a first fluid space S1 of the housing 810 from a second fluid space S2 of the housing 810. As used herein, the first fluid space S1 is “fluidly isolated” from the second fluid space S2 in the sense that the fluid spaces S1, S2 are not in fluid communication so respective fluid pressures in each fluid space S1, S2 do not spontaneously equilibrate. In some embodiments, the first fluid space S1 may be open to a surrounding environment and have a fluid pressure that is substantially similar to atmospheric pressure. The movable seal 850, which may be a stopper disposed in a container holding one or more drug products, fluidly isolates a third fluid space S3 from the second fluid space S2, the significance of which will be described further herein.

The release mechanism 840 may be disposed in a release mechanism opening 826 formed in the partition 825 and at least partially surrounded by, or embedded within, a plug 841 that seals the release mechanism opening 826 and maintains fluid isolation between the first fluid space S1 and the second fluid space S2. In some embodiments, an indicator assembly 820, which may be similar to any of the previously described indicator assemblies, is disposed in the first fluid space S1 of the housing 810 and sealed off from the second fluid space S2 of the housing 810 in which the movable seal 850 is disposed. The release mechanism 840 may be linked to the movable seal 850 by a tensioner 870, which may be a monofilament wire or similar element, such that movement of the movable seal 850 to an injection end position causes the release mechanism 840 to release the indicator assembly 820. The injection device 800 further includes a valve 890 that extends into the second fluid space S2 of the housing 810 to, for example, selectively inject pressurized fluid into the second fluid space S2 of the housing 810, increasing the fluid pressure in the second fluid space S2 and driving the movable seal 850 when the valve 890 is activated.

When the valve 890 activates, pressurized fluid enters the second fluid space S2 of the housing 810 and urges the movable seal 850 toward the injection end position, As the movable seal 850 moves toward the injection end position, the movable seal 850 pressurizes the third fluid space S3 to urge drug product from the housing 810 and pulls the release mechanism 840 via the tensioner 870, Once the movable seal 850 reaches the injection end position, the release mechanism 840 releases the indicator assembly 820 to a dose end position so a user may see that the injection has ended, In some embodiments, the movable seal 850 fully pulls the release mechanism 840 through the release mechanism opening 826 upon reaching the injection end position, establishing a path for the fluid pressure in the second fluid space S2 to vent and equalize with the pressure in the first fluid space S1. In some embodiments, the burst of fluid venting into the first fluid space S1 may partially or fully drive the indicator assembly 820 to the dose end position. Alternatively, in some embodiments, at least a portion of the release mechanism 840 remains in the release mechanism opening 826 upon the movable seal 850 reaching the injection end position, maintaining the fluid separation between the first fluid space S1 and the second fluid space S2.

Referring now to FIGS. 8B-8D, exemplary embodiments of the partition 825 illustrated in FIG. 8A for fluidly isolating the first fluid space S1 and the second fluid space S2 are illustrated. In one embodiment, illustrated in FIG. 8B, a partition 825A is formed as a septum comprising a rubber or similar material with the tensioner 870, which may be a monofilament wire, pierced through the septum 825A by, for example, a needle or other sharp element carried by the tensioner 870 to form a tensioner opening 872 in the septum 825A. The rubber material of the septum 825A seals around the tensioner 870 in the tensioner opening 872 to seal and isolate the first fluid space S1 from the second fluid space S2.

In another embodiment, illustrated in FIG. 8C, a partition 825B is formed as a septum comprising a rubber or similar material with the tensioner 870 extending through a tensioner opening 871 formed in the septum 825B and sealed with a sealant or other type of material.

In another embodiment, illustrated in FIG. 8D, a partition 825C is formed as a rolling diaphragm seat that seals around the tensioner 870 to maintain the fluid isolation between the first fluid space S1 and the second fluid space S2.

It should be appreciated from the foregoing that indicator assemblies disclosed herein may be used to indicate an injection has ended in injection devices that utilize pressurized fluid, rather than solid elements, to drive the injection. Thus, the indicator assemblies disclosed herein may be incorporated and function in many different types of injection devices, unlike many conventional indicators.

While the previously described injection devices 100, 300, 400, 500, 600, 800 are configured so their respective indicators are binary EOD indicators, in some embodiments the injection devices 100, 300, 400, 500, 600, 800 are configured so their respective indicators convey injection progress.

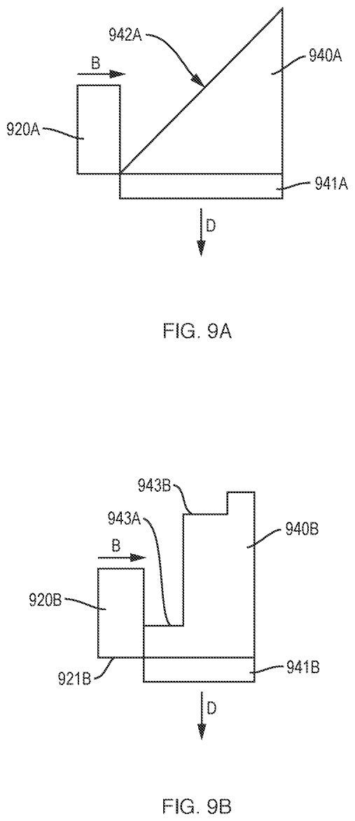

To convey progressive injection progress, and referring now to FIG. 9A, an injection device may include a release mechanism that allows progressive movement of an indicator assembly to the dose end position as the linked movable seat moves toward the injection end position, in one exemplary embodiment illustrated in FIG. 9A, the release mechanism includes a pin 940A with a tapered surface 942A held in a release mechanism opening 941A. The tapered surface 942A abuts against and interferes with movement of a portion 920A of an indicator assembly, which includes one or more dose end indicators and is biased in a biasing direction B toward a dose end position. As the pin 940A moves in a distal direction D, the portion 920A progressively slides along the tapered surface 942A until the portion 920A clears an entirety of the pin 940A to reach the dose end position. The one or more dose end indicators of the portion 920A become progressively viewable as the portion 920A progressively slides along the tapered surface 942A, allowing a user to follow progress of the injection.

To convey step-wise injection progress, and referring now to FIG. 9B, an injection device may include a release mechanism that allows step-wise movement of an indicator assembly to the dose end position as the linked movable seal moves toward the injection end position. In one exemplary embodiment illustrated in FIG. 9B, the release mechanism includes a stepped pin 940B with a series of steps 943A, 943B held in a release mechanism opening 941B. One of the steps, such as step 943A, initially abuts against and interferes with movement of a portion 920B of an indicator assembly, which includes one or more dose end indicators and is biased in a biasing direction B toward a dose end position. As the stepped pin 940B moves in a distal direction D, the steps 943A, 943B move in the distal direction D. When a step, such as step 943A, travels in the distal direction D below a bottom 921B of the portion 920B, the portion 920B clears the step 943A and moves in the biasing direction B toward the dose end position until the portion 920B contacts the next step 943B, which inhibits further progress of the portion 920B. This step-wise movement of the portion 920B may repeat until the portion 920B clears all of the steps of the stepped pin 940B and releases to the dose end position. It should be appreciated that the number and shape of the steps 943A, 943B may be altered to provide the desired step-wise movement of the indicator assembly toward the dose end position.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step to may be replaced with a plurality of elements or steps that serve the same purpose. Further, where parameters for various properties are specified herein for exemplary embodiments, those parameters may be adjusted up or down by 1/20th, 1/10th, ⅕th, ½nd, and the like, or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention, Further still, other aspects, functions and advantages are also within the scope of the invention.

1. (canceled)

2. A method of indicating an end of dose in an injection device, the method comprising:
displacing a movable seal in an internal volume of the injection device from an initial position to an injection end positon to urge a drug product out of the injection device;
where a dose end indicator is visible through a viewing portion of a housing of the injection device, and the indicator assembly includes the dose end indicator;
holding the indicator assembly in the dose remaining position; and
releasing the indicator assembly from the dose remaining position.

3. The method of indicating an end of dose in the injection device of claim 2, wherein the indicator assembly is pivotably biased toward the dose end position.

4. The method of indicating an end of dose in the injection device of claim 2, further comprising:
biasing the indicator assembly toward the dose end position using a biaser bearing on the indicator assembly.

5. The method of indicating an end of dose in the injection device of claim 2, wherein the indicator assembly includes:
a first portion that is visible through the viewing portion when the indicator assembly is in the dose remaining position; and
a second portion that is visible through the viewing portion when the indicator assembly is in the dose end indicator.

6. The method of indicating an end of dose in the injection device of claim 2, wherein the housing has a proximal end and a distal end opposite the proximal end.

7. The method of indicating an end of dose in the injection device of claim 6, wherein displacing the movable seal to the injection end position dislodges a release mechanism out of engagement with the indicator assembly, the injection end position of the movable seal is distal to the initial position.

8. The method of indicating an end of dose in the injection device of claim 2, further comprising a tensioner linking the movable seal to the release mechanism.

9. The method of indicating an end of dose in the injection device of claim 8, further comprising stopping the indicator assembly from moving past the end of dose position in response to the second portion hitting a stop adjacent to the indicator assembly.

10. The method of indicating an end of dose in the injection device of claim 2, further comprising a release mechanism that causes releasing of the indicator assembly from the dose remaining position responsive to the release mechanism being triggered.

11. The method of indicating an end of dose in the injection device of claim 10, the method further comprising:
holding the indicator assembly in the dose remaining position in response to the release mechanism abutting against a contact surface of a dome, the dome including a first portion and a second portion of the indicator assembly.

12. An injection device, comprising:
a housing having an internal volume and a viewing portion;
a movable seal disposed in the internal volume that is movable from an initial position to an injection end position to urge a drug product out of the housing;
an indicator assembly disposed in the internal volume and including a dose end indicator, the indicator assembly being biased from a dose remaining position toward a dose end position where the dose end indicator is visible through the viewing portion; and
a release mechanism releasably holding the indicator assembly in the dose remaining position and configured to release the indicator assembly to the dose end position when triggered.

13. The injection device of claim 12, wherein the indicator assembly is pivotably biased toward the dose end position.

14. The injection device of claim 13, further comprising a biaser bearing on the indicator assembly to bias the indicator assembly toward the dose end position.

15. The injection device of claim 12, wherein the indicator assembly includes a first portion that is visible through the viewing portion when the indicator assembly is in the dose remaining position and a second portion including the dose end indicator.

16. The injection device of claim 13, wherein the housing has a proximal end and a distal end opposite the proximal end.

17. The injection device of claim 16, wherein the injection end position of the movable seal is distal to the initial position and the movable seal moving distally to the injection end position pulls the release mechanism out of engagement with the indicator assembly.

18. The injection device of claim 12, further comprising a tensioner linking the movable seal to the release mechanism.

19. The injection device of claim 12, further comprising a partition dividing the internal volume into a first fluid space and a second fluid space, where the second fluid space is fluidly isolated from the first fluid space.

20. The injection device of claim 19, wherein the indicator assembly is disposed in the first fluid space and the movable seal is disposed in the second fluid space.

21. The injection device of claim 20, further comprising a valve in fluid communication with the second fluid space for selectively increasing a fluid pressure in the second fluid space to urge the movable seal toward the injection end position.
